(19) United States
(12) Patent Application Publication
(10) Pub. No.: US 2026/0346029 A1
(43) Pub. Date: Nov. 5, 2026

(54) Implantable Medical Device Allowing a Simplified Fixation

(71) Applicant: BIOTRONIK SE & Co. KG, Berlin (DE)

(72) Inventors: Ingo WEISS, Pforzheim (DE); Thomas DOERR, Berlin (DE); Luise ROGGE, Berlin (DE)

(73) Assignee: BIOTRONIK SE & Co. KG, Berlin (DE)

(21) Appl. No.: 19/486,856

(22) PCT Filed: May 29, 2024
(86) PCT No.: PCT/EP2024/064726

§ 371 Date: Nov. 21, 2025

(30) Foreign Application Priority Data
Jun. 7, 2023 (EP) 23177926.5

(51) Int. Cl. A61N 1/375

(52) U.S. Cl. CPC A61N 1/37518

(57) ABSTRACT

The invention relates to an implantable medical device comprising a housing. The housing comprises a slit, wherein the slit separates the housing into a first housing part and a second housing part, wherein at least one of the first housing part and the second housing part comprises at least one constriction. The slit and the at least one constriction are designed and arranged such that a thread for fixing the implantable medical device to a body structure can be guided through the slit and i) around the first housing part in a region of the constriction and/or ii) around the second housing part in a region of the constriction, and can be fixed at the first housing part and/or the second housing part by forming a sling around the first housing part and/or the second housing part.

### CROSS-REFERENCE TO RELATED APPLICATIONS

This application is the United States National Phase under 35 U.S.C. § 371 of PCT International Patent Application No. PCT/EP 2024/064726, filed on May 29, 2024, which claims the benefit of European Patent Application No. 23177926.5, filed on Jun. 7, 2023, the disclosures of which are hereby incorporated by reference herein in their entireties.

### TECHNICAL FIELD

The present invention relates to an implantable medical device according to the preamble of claim 1, and to an arrangement of an implantable medical device according to claim 14.

### BACKGROUND

Implantable medical devices, for instance implantable electrotherapeutic devices, need to be fixed to a body structure such as body tissue in order to stay in place after implantation. For this purpose, implantable medical devices known from prior art comprise a hole through which a surgical needle carrying a thread for fixing the implantable medical device can be guided. Since these surgical needles are typically bent, the respective hole in the implantable medical device needs to be quite big. This requires a lot of space in the implantable medical device. Additionally, a surgeon needs to locate the hole with the surgical needle. This task is particularly complicated if the implantable medical device is to be fixed with a suture within a hard to reach soft tissue pocket.

### SUMMARY

It is an object of the present invention to provide an implantable medical device that can be easier fixed to a body part than implantable medical devices known from prior art.

This object is achieved with an implantable medical device having the features of claim 1. Such a device comprises a housing. According to an aspect of the present invention, this housing comprises a slit. This slit separates the housing into a first housing part and a second housing part. In this context, the first housing part and/or the second housing part comprises at least one constriction. The slit and the constriction in the first and/or second housing part form a fixing element that is integrated into the housing and that is much easier accessible than a hole through which surgical needle is to be guided. The slit and the constriction are designed and arranged such that a thread (in particular surgical thread) for fixing the implantable medical device to a body structure such as body tissue can be guided through the slit. Furthermore, the thread can be guided around the first housing part in the region of the constriction (if the first housing part comprises such constriction) and/or around the second housing part in a region of the constriction (if the second housing part comprises such constriction). Furthermore, the thread can be fixed at the first housing part and/or at the second housing part by forming a sling around the first housing part and/or the second housing part (depending on which housing part the constriction is formed). Due to this arrangement of a slit and a constriction in a housing part being located adjacent to the slit, it is no longer necessary to guide a surgical needle through a hole of the implantable medical device. In fact, the implantable medical device does no longer require such a hole and has, in an embodiment, no such hole for allowing a surgical needle to pass through. Rather, the slit offers a much more flexible possibility to receive a surgical thread than the fixing holes known from prior art do. Therefore, it is much easier for a surgeon to find the slit with the thread to be used for fixing the implantable medical device to body tissue (or other body parts).

The constriction prevents an undesired slipping of the thread off the first housing part or the second housing part once the thread has been guided around the first housing part and/or the second housing part. Thus, the constriction helps in securing a thread around the first housing part and/or the second housing part and serves for a stable support of a thread that is guided around the first housing part and/or the second housing part.

While a surgical needle is typically still necessary in order to form a suture into body tissue for fixing the implantable medical device, it is no longer necessary to guide the needle through any part of the implantable medical device. Rather, for fixing the implantable medical device, a surgeon will fix a thread on a surgical needle (or will use a surgical needle already carrying a thread) and will then guide the thread into the slit (essentially perpendicular to a longitudinal extension direction of the thread). The surgeon will guide the thread furthermore around the first housing part and/or the second housing part in the region of the constriction and form a sling with the thread around the first housing part and/or the second housing part in this region. Alternatively, a sling may be formed prior to guiding the thread into the slit, e.g. already outside the patient's body. Once the thread forms a sling around the first housing part and/or the second housing part, the thread is already fixed to the implantable medical device, whereupon the surgeon can perform his usual suturing technique for fixing the thread to a body part and thus fixing the implantable medical device that is already connected to the thread with that body part.

Since the slit requires much less space than a hole for receiving a surgical needle, the implantable medical device can be manufactured in a space-saving manner. Due to a smaller volume of the implantable medical device, more devices can be implanted in a minimally invasive way, resulting in an increased patient comfort. The surgeon will be able to fix the implantable medical device much faster to a body part of the patient to whom the implantable medical device is implanted. Therefore, the implantation procedure will require less time. Furthermore, the fixing procedure itself can be accomplished in a much easier way, resulting in a better usability of the implantable medical device for the surgeon. Even if the implantable medical device is to be fixed within a hard to reach soft tissue pocket, the fixing procedure can be accomplished much easier than according to prior art techniques since no bent surgical needle needs to be guided through a hole within the implantable medical device.

According to an advantageous embodiment of the present invention, the implantable medical device is an implantable electrotherapeutic device.

In an embodiment, the slit has a constant height. In this context, the height is defined as distance between the first housing part and the second housing part in a region in which the slit is formed. A housing with the slit having a constant height can be particularly easy constructed. The walls of the first housing part and the second housing part facing the slit run parallel to each other to achieve such a constant height. In an embodiment, the height is smaller than 2 mm, in particular smaller than 1 mm, in particular smaller than 0.5 mm. In an embodiment, the height lies in a range of from 0.01 mm to 2 mm, in particular from 0.05 mm to 1.5 mm, in particular from 0.1 mm to 1 mm, in particular from 0.5 mm to 0.8 mm.

In an embodiment, the slit has a varying height in a longitudinal direction of the slit, i.e., along a depth of the slit. As already outlined above, the height is defined as distance between the first housing part and the second housing part in a region in which the slit is formed. Such a varying height can be achieved if the walls of the first housing part and of the second housing part facing the slit run, at least in a section, not parallel to each other. By such a different height along the length or depth of the slit, a contour of the slit can be realized that suits the needs for enabling an easy introduction of a thread into the slit as well as a safe fastening of a thread around the first housing part or the second housing part in the region of the constriction.

In an embodiment, the height of the slit decreases from a frontal opening (face side opening) of the slit to a bottleneck section of the slit. In such a case, the frontal opening allows an easy introduction of a thread into the slit, wherein the contour of the slit guides such an inserted thread towards the bottleneck section of the slit. This decrease of the height is, in an embodiment, a constant decrease. In another embodiment, the height of the slit decreases and increases at least in a section to subsequently further decrease until the bottleneck section of the slit is reached.

In an embodiment, the bottleneck section is positioned, in a longitudinal direction of the slit, distally of the frontal opening of the slit and proximally of a distal terminus of the slit. Expressed in other words, the bottleneck section does, in this embodiment, not constitute the most distal region of the slit, but is rather arranged in an intermediate section of the slit. Then, the height of the slit increases again after the bottleneck section, wherein the height of the slit proximally to the bottleneck section and distally to the bottleneck section can be independently chosen from each other. In an embodiment, the at least one constriction is arranged in a section of the slit that lies distally of the bottleneck section.

In an embodiment, a wall of the first housing part facing the slit and a wall of the second housing part facing the slit contact each other in the bottleneck section. Then, it is only possible to guide a thread across the bottleneck section by elastically deforming at least one of the wall of the first housing part and the wall of the second housing part. In addition, the thread is typically also elastically deformed upon such introduction procedure. Once the thread has been guided across the bottleneck section, the contact between the wall of the first housing part and the wall of the second housing part effects that the thread cannot be slid off the slit towards the frontal opening of the slit again. Then, the thread can be particularly easy guided around the first housing part and/or the second housing part in the region of the constriction.

In an embodiment, a first barbed hook for a thread to be inserted into the slit is formed in the bottleneck section. By such a barbed hook, a movement of a thread within the slit in a longitudinal direction of the slit can be particularly easy allowed in a first direction (towards the distal terminus of the slit) and prohibited in a second direction (towards the proximal frontal opening of the slit). Thus, the barbed hook is typically used for allowing an easy introduction of a thread into the slit from the frontal opening of the slit towards a distal (rear) position of the slit, but prevents a movement of a thread that has been inserted into the slit beyond the barbed hook back to a section of the slit lying before or proximally to the barbed hook.

In an embodiment, the first housing part and/or the second housing part comprises a recess for receiving a thread. In this context, the recess is formed adjacent to the constriction. Moreover, the recess may be formed as an undercut in the longitudinal direction. In particular, the recess may be formed between the frontal opening of the slit and the constriction and/or between the constriction and the terminus of the slit. Then, a thread that is guided around the constriction can be slid into the recess. The recess serves-together with a restriction-for a particularly secure fastening possibility of a thread around the first housing part and/or the second housing part.

In an embodiment, the walls defining the recess form a bottleneck section of the recess. On the one hand, this can facilitate guiding the thread into the recess. On the other hand, this can prevent a thread that has been inserted into the recess from unintentionally leaving the recess again.

In an embodiment, the first housing part and/or the second housing part comprises a second barbed hook that is formed between the constriction and the recess. By such a second barbed hook, a movement of a thread from the restriction into the recess can be made particularly easy, whereas a movement back out of the recess is efficiently prevented by the second barbed hook. Like the first barbed hook, also the second barbed hook can give a tactile response to surgeon guiding a thread through the slit and into the recess of the implantable medical device so that the surgeon is aware of the position at which the thread is actually located, even though no visual control is possible during this procedure.

In an embodiment, the first housing part and the second housing part are formed from one piece. Then, the slit is typically introduced into the housing upon manufacturing of the housing or after having manufactured the housing. In this embodiment, the slit can be realized, e.g., by a cutting into the housing.

In another embodiment, the second housing part is mounted onto the first housing part. This mounting can be realized, e.g., by welding, gluing, press joining, riveting and/or screwing the second housing part onto the first housing part. Also a snap-fit connection between the first housing part and the second housing part can be particularly easy realized to mount the second housing part onto the first housing part. Such a snap-fit connection can be assisted by additionally gluing the housing parts together.

In an embodiment, the first housing part and/or the second housing part are made from a metal, plastic and/or a ceramic. If the first housing part and the second housing part are formed from one piece, metal (such as a biocompatible stainless steel or titanium) is a particularly appropriate material for producing the housing. If the second housing part is mounted onto the first housing part, the first housing part (which typically constitutes the bigger part of the housing) is, in an embodiment, made from a metal such as stainless steel or titanium. At the same time, the second housing part is, in this embodiment, made from a plastic. In this context, an elastically deformable plastic is a particularly appropriate material for the second housing part.

In an embodiment, the first and/or the second housing part, in particular the smaller one of the first and second housing part has a volume of equal to or less than 2cm³ , between 2 cm³ and 1 cm³, equal to or less than 1 cm³ or equal to or less than 0.5 cm³.

In an embodiment, the second housing part is made from a liquid crystal polymer (LCP), a polysulfone (PSU), a polyetherimide (PEI), a polyether ether ketone (PEEK), a polyphenylsulfone (PSU), an epoxy resin, titanium, and/or stainless steel. It should be acknowledged that any of these materials are also appropriate for forming the first housing part. As outlined above, the first housing part and the second housing part may be made of the same material or may be made from different materials.

In an aspect, the present invention relates to an arrangement of an implantable medical device according to the preceding explanations as well as a thread for fixing the implantable medical device to a patient's body structure such as patient's body tissue. The thread may already be guided into the slit of the implantable medical device and optionally around the first housing part and/or the second housing part or may be provided in a condition in which it is not yet introduced into the slit. Such an arrangement makes it particularly easy to fix the implantable medical device to the patient's body structure, as explained above in more detail.

In an aspect, the present invention relates to medical method for fixing an implantable medical device according to the preceding explanations to a body structure of a patient in need thereof. This method comprises the steps explained in the following.

In one method step, a thread is guided through the slit formed in the housing of the implantable medical device. In this context, the slit separates the housing into a first housing part and a second housing part.

In a further method step, the thread is guided around constriction formed in at least one of the first housing part and the second housing part.

Prior to this or afterwards, a sling is formed with the thread that extends around the first housing part and/or the second housing part in a region of the constriction.

In another method step, the thread is secured to a body structure of the patient. It should be noted, that this the individual method steps can be performed in a different sequence. In particular, the method step explained last can also be done at a different stage of the precedingly explained method. To give an example, the thread may be first secured to a body structure of the patient and only afterwards fixed at the implantable medical device by carrying out the other method steps.

As explained above, this method of fixing an implantable medical device to a body structure can be accomplished significantly faster and easier than fixing methods known from prior art that require the guiding of a surgical needle through a hole formed in an implantable medical device.

All embodiments of the implantable medical device can be combined in any desired way and can be transferred either individually or in any arbitrary combination to the arrangement and to the method. Furthermore, all embodiments of the arrangement can be combined in any desired way and can be transferred either individually or in any arbitrary combination to the implantable electrotherapeutic device and to the method. Finally, all embodiments of the method can be combined in any desired way and can be transferred either individually or in any arbitrary combination to the implantable medical device and to the arrangement.

### BRIEF DESCRIPTION OF THE DRAWINGS

Further details of aspects of the present invention will be explained in the following making reference to exemplary embodiments and accompanying Figures. In the Figures:

FIG. 1 shows a detail of a prior art implantable medical device;

FIG. 2A shows a lateral view of a detail of a first embodiment of an implantable medical device;

FIG. 2B shows a perspective view onto the implantable medical device of FIG. 2A;

FIG. 2C shows a lateral view of a detail of a second embodiment of an implantable medical device;

FIG. 2D shows a perspective view onto the implantable medical device of FIG. 2C;

FIG. 2E shows a lateral view of a detail of a third embodiment of an implantable medical device;

FIG. 2F shows a perspective view onto the implantable medical device of FIG. 2E;

FIG. 2G shows a top view onto the implantable medical device of FIG. 2C;

FIGS. 2H and 2I illustrate the dimensions of the slit formed in an embodiment of an implantable medical device;

FIG. 2J illustrates the positioning of a thread around a housing part of an embodiment of an implantable medical device;

FIG. 3A shows a schematic top view of a first embodiment of a second housing part of an implantable medical device;

FIG. 3B shows a schematic top view of a second embodiment of a second housing part of an implantable medical device;

FIG. 3C shows a schematic top view of a third embodiment of a second housing part of an implantable medical device;

FIG. 3D shows a schematic top view of a fourth embodiment of a second housing part of an implantable medical device;

FIG. 4 shows a schematic top view of another embodiment of a housing of an implantable medical device;

FIG. 5A shows a schematic cross-sectional view through a further embodiment of a housing of an implantable medical device;

FIG. 5B shows a schematic cross-sectional view through a further embodiment of a housing of an implantable medical device; and

FIG. 5C shows a schematic cross-sectional view through a further embodiment of a housing of an implantable medical device.

### DETAILED DESCRIPTION

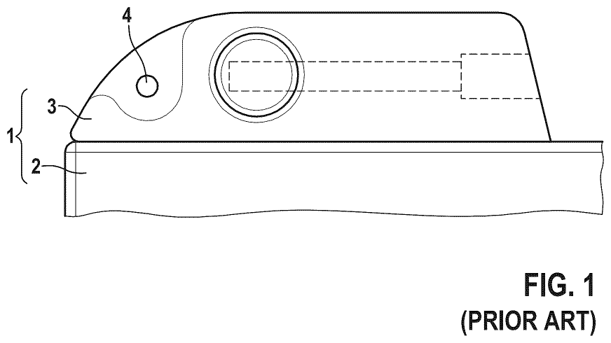

FIG. 1 shows lateral view of a housing 1 of an implantable medical device, in particular an implantable electrotherapeutic device known from prior art. This housing 1 comprises a lower housing part 2 and a header 3 arranged on top of the lower housing part 2. The header 3 serves for connecting an electrode lead with a circuitry arranged within the housing 1. For fixing the housing 1 to a patient's body tissue, the header 3 comprises a hole 4 through which a surgical thread can be guided with a surgical needle. Since surgical needles are typically bent, the hole 4 is comparatively big to allow a restriction-less guiding of the needle through the hole 4. Consequently, it is necessary to provide a sufficient amount of material around the hole 4 to maintain the integrity of the header 3. This results in a non-negligible space requirement for providing the hole 4 in the header 3. Even then, it may be quite complicated for a surgeon to locate the hole 4 and to guide a surgical needle through the hole 4 if the housing 1 is already positioned at its intended site of implantation. All in all, fixing the housing 1 and thus the implantable electrotherapeutic device by guiding a needle and a thread through the hole 4 is a laborious procedure requiring skill, training and time.

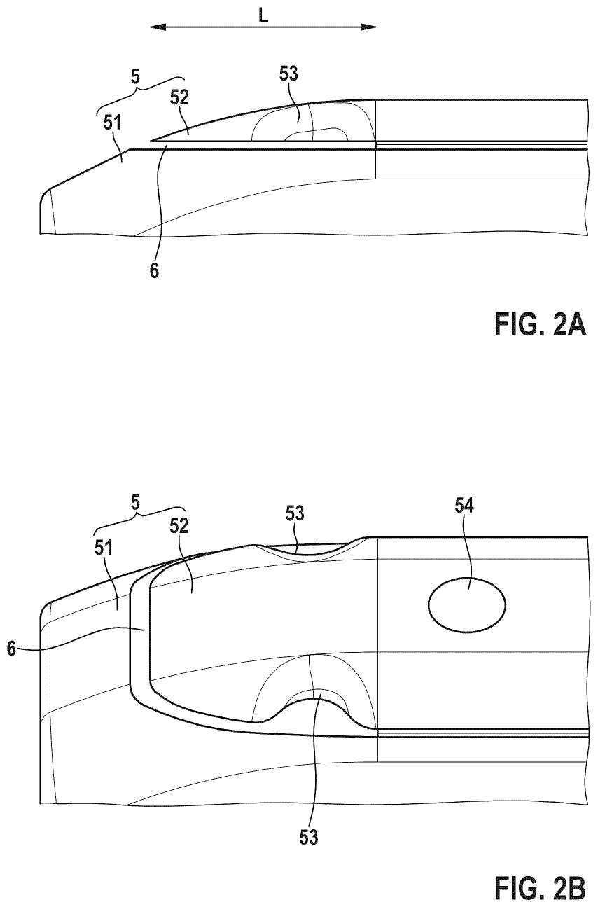

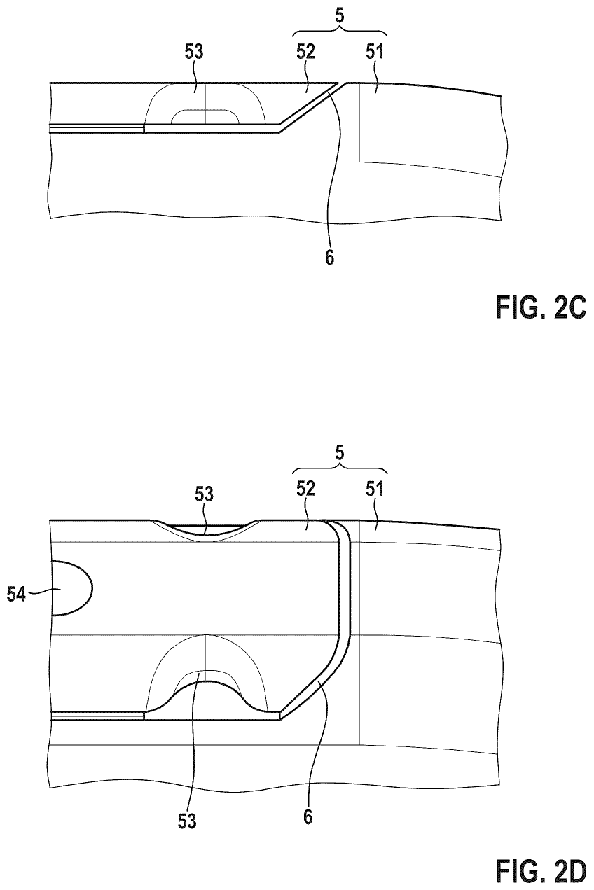

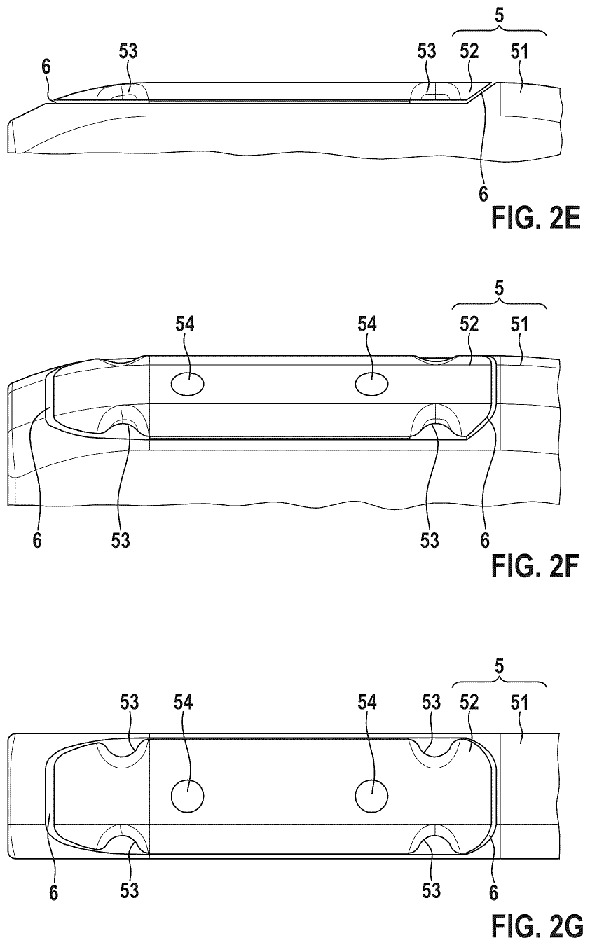

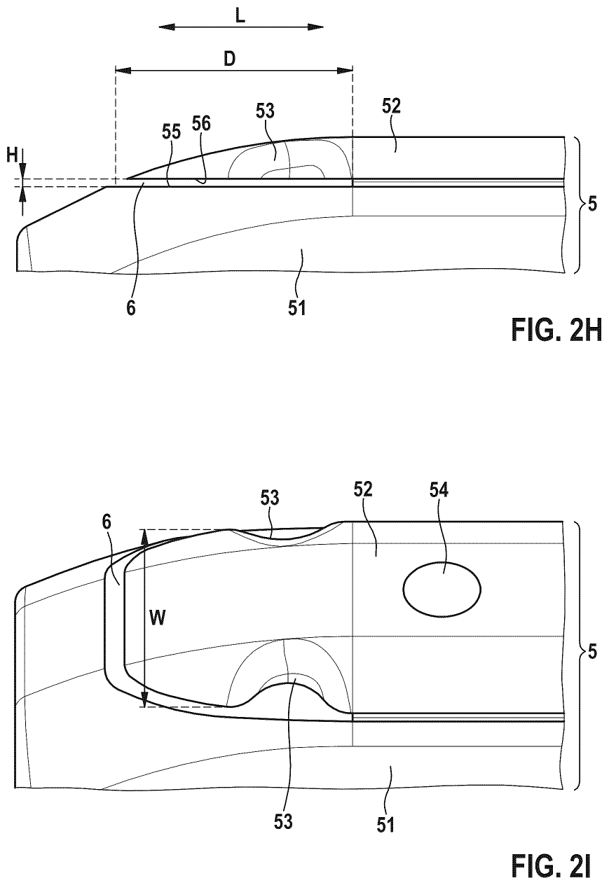

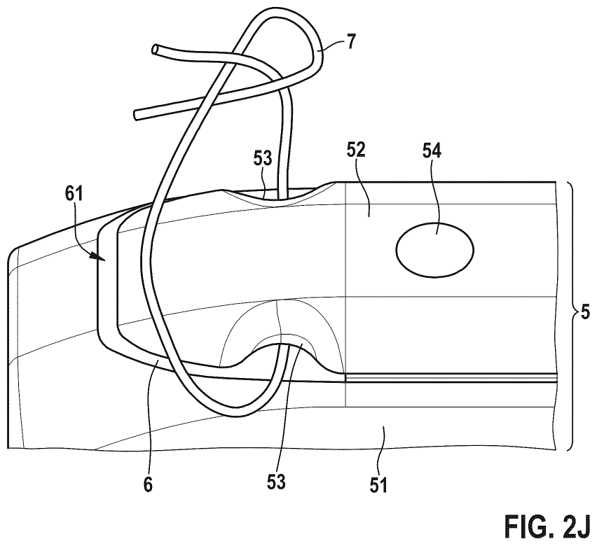

FIG. 2A is a lateral view of a detail of a first embodiment of an implantable medical device which for instance, can be an implantable electrotherapeutic device. This implantable medical device comprises a housing 5 that, in turn, comprises a slit 6 that divides the housing 5 into a first housing part 51 and a second housing part 52. The slit 6 extends horizontally in a longitudinal direction L into the housing 5. The second housing part 52 is designed as a top cover. It comprises a constriction 53 in a width direction perpendicular to the longitudinal direction L. The constriction 53 can be even better seen in the perspective view of FIG. 2B.

In this and in all following Figures, similar elements will be denoted with the same numeral reference. The constriction 53 serves for keeping a thread in place that has been inserted into the slit 6 and that is guided in the width direction around the second housing part 52 to be secured to the housing 5.

The second housing part 52 is mounted onto the first housing part 51 by a snap-fit mechanism realized with the help of a pin 54 that forms an integral part of the first housing part 51. The second housing part 52 is snapped onto this pin 54 and can be additionally fixed with glue.

FIG. 2C shows a side view of a second embodiment of a housing 5 of an implantable medical device such as an implantable electrotherapeutic device. The general setup of the housing 5 is identical to the setup illustrated in FIGS. 2A and 2B. However, the slit 6 does not extend horizontally into the housing 5, but rather has an angled course. Nonetheless, it separates a first housing part 51 from a second housing part 52 and allows the insertion of a thread for fixing the housing 5 onto patient's body tissue. The second housing part 52 comprises a constriction 53, as already explained with respect to the first embodiment shown in FIGS. 2A and 2B.

FIG. 2D shows a perspective view onto the housing 5 of the implantable therapeutic device of FIG. 2C. Here, it can once again be seen that the second housing part 52 is mounted onto the first housing part 51 with a snap-fit mechanism taking advantage of a pin 54 that forms an integral part of the first housing part 51 and is, in the mounted state of the housing 5, surrounded by the second housing part 52.

FIG. 2E shows another embodiment of a housing 5 of an implantable medical device that is a combination of the embodiments shown in FIGS. 2A and 2B on the one hand and in FIGS. 2C and 2D on the other hand. In the lateral view of FIG. 2E, it can well be seen that the housing 5 comprises a first slit 6 on its front or proximal region (oriented to the left in FIG. 2E), where a first constriction 53 is disposed, as well as a second slit 6 in its rear or distal region (oriented to the right in FIG. 2E), where a second constriction 53 is disposed. These slits 6 are not connected to each other. Rather, they reach only into the housing 5 up to the region of the respective constriction 53.

The embodiment shown in FIG. 2E allows a fixation of the housing 5 of the implantable medical device with two different threads. One thread can be inserted into the front slit 6 and guided around the front constriction 53, whereas another thread can be inserted into the rear slit 6 and guided around the rear constriction 53.

FIG. 2F shows a perspective view of the housing 5 FIG. 2E, whereas FIG. 2G shows a top view onto the housing 5 of FIG. 2E. Reference is made to the explanations given above with respect FIGS. 2A to 2C regarding the construction of the housing.

FIGS. 2H and 2I illustrate the dimensions of the slit 6 within the housing 5. The slit 6 has a height H that is defined by a distance between the first housing part 51 and the second housing part 52, or, to be more precise, between a first wall 55 of the first housing part 51 and a second wall 56 of the second housing part 52, wherein the first wall 55 and the second wall 56 face the slit 6.

The slit 6 furthermore has a depth D extending in the longitudinal direction L of the slit 6. As can be seen from FIG. 2I, the slit 6 furthermore has a width W that reaches from a first side to a second side of the housing 5.

FIG. 2J illustrates an arrangement of an implantable medical device and a thread 7 guided around the second housing part 52 of the implantable medical device. For this purpose, the thread 7 is inserted into the slit 6 from a frontal opening 61 of the slit 6 up to the region of the constriction 53 of the second housing part 52. Then, the thread 7 is guided around the second housing part 52 to form a sling around the second housing part 52. Afterwards, the thread 7 can be sutured to the patient's body tissue to safely secure the implantable medical device to the patient's body tissue.

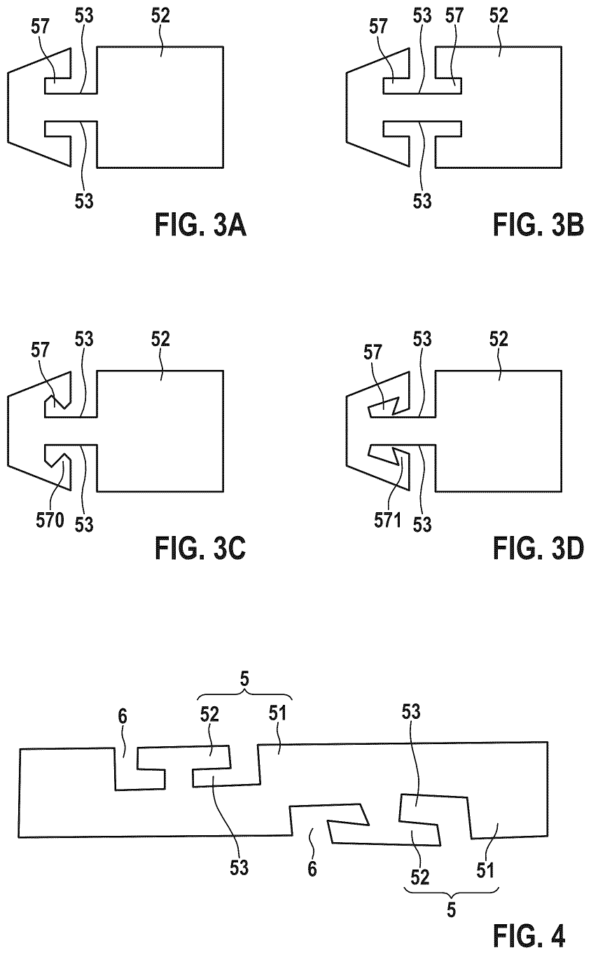

FIG. 3A shows a schematic top view depiction of a first embodiment of the second housing part 52, in which the second housing part 52 does not only comprise the constriction 53, but in addition a recess 57. If a thread is guided through the slit and around the second housing part 52 in the region of the constriction 53, it can be furthermore guided into the recess 57 to be even better protected against an undesired repositioning. This facilitates fixing the housing 5 to a body structure and ensures a particularly stable positioning of the housing 5 being fixed with the thread located within the recess 57.

The recess 57 can be arranged towards a (proximal) terminal side of the second housing part 52, as illustrated in FIG. 3A, to a central portion of the housing 52 (not separately illustrated), or both to a terminal side and a central portion of the second housing part 52, as illustrated in FIG. 3B.

To reduce the risk that a thread that has been guided into the recess 57 unintentionally leaves the recess 57 once again, the walls of the second housing part 52 surrounding the recess 57 can form a bottleneck portion 570 between the recess 57 and the constriction 53. This embodiment is illustrated in FIG. 3C.

In a further embodiment, which is illustrated in FIG. 3D, the walls of the second housing part 52 that surround the recess 57 do not only form a bottleneck section, but rather a barbed hook 571. Such a barbed hook 571 particularly efficiently prevents a thread that has been guided from the constriction 53 into the recess 57 to leave the recess 57 once again unintentionally.

In the embodiments illustrated in FIGS. 3A to 3D, the second housing part 52 has, in its terminal region, an anchor-like shape given by the constriction 53, the recess 57 and the wall surrounding the recess 57 and the constriction 53. Obviously, this embodiment cannot only be realized as a further development of the embodiment shown in FIGS. 2A and 2B, but also as a further development of the embodiments shown in FIGS. 2C to 2G.

FIG. 4 shows another embodiment of the housing 5 of an implantable medical device which allows a lateral fixation of a thread to the housing 5. In this context, FIG. 4 is a top view onto the housing 5, wherein the element seen in FIG. 4 can be realized as cover plate to be connected to a bigger housing part, if desired. In this embodiment, a thread can be particularly easy inserted into the slit 6 towards the recess 53 and being secured around the second housing part 52. In some instances, a lateral fixation as made possible by the embodiment shown in FIG. 4 may be more preferable than a top-side fixation as illustrated in the above-described embodiments.

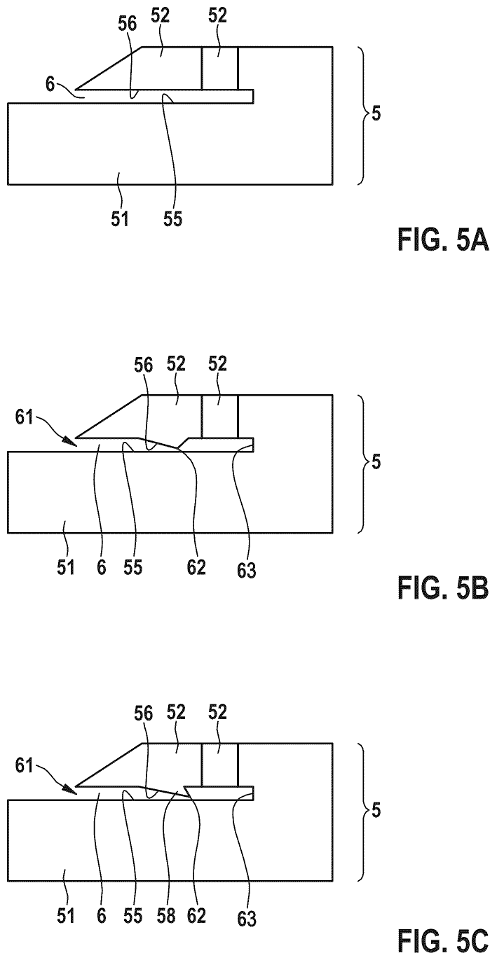

FIGS. 5A, 5B, and 5C show schematic cross-sectional views through different embodiments in which the shape of the slit 6 within the housing 5 is realized in a different way. In FIG. 5A, the slit 6 has a constant height, i.e., the first wall 55 of the first housing part 51 and the second wall 56 of the second housing part 52 extend parallel to each other. In the embodiment shown in FIG. 5B, the first wall 55 and the second wall 56-at least section-wise—do not extend parallel to each other. Consequently, a bottleneck section 62 of the slit 6 is formed by the cooperation of the first wall 55 and the second wall 56. The bottleneck section 62 of the slit 6 is distal to the frontal opening 61 of the slit 6, but proximal to a distal terminus 63 of the slit 6. If a thread is inserted into the slit 6, it needs to pass the bottleneck section 62 of the slit 6 to reach the region of the constriction 53 of the second housing part 52. Once the thread has reached this region, it will be difficult for the thread to unintentionally leave its position once again towards the frontal opening 61 of the slit 6.

FIG. 5C shows an embodiment of the housing 5 in which a barbed hook 58 is formed by the second wall 56 of the second housing part 52 in the bottleneck section 62 of the slit. This barbed hook 58 makes it even more unlikely that a thread that has passed the bottleneck section 62 of the slit 6 exits the slit 6 again through the frontal opening 61 of the slit 6. Rather, will be kept safely in place in the region in which the constriction 53 is formed within the second housing part 52.

It should be noted that the bottleneck section 62 and the barbed hook 58 do not necessarily extend over the whole width W of the slit 6 (cf. FIG. 2I for more details). Rather, it is fully sufficient, if the bottleneck section 62 and/or the barbed hook 58 only extend over a section of the width W of the slit 6.

1. Implantable medical device comprising a housing,
wherein the housing comprises a slit,
wherein the slit separates the housing into a first housing part and a second housing part,
wherein at least one of the first housing part and the second housing part comprises at least one constriction
wherein the slit and the at least one constriction are designed and arranged such that a thread for fixing the implantable electrotherapeutic device to a body structure can be guided through the slit and i) around the first housing part in a region of the constriction and/or ii) around the second housing part in a region of the constriction, and can be fixed at the first housing part and/or the second housing part by forming a sling around the first housing part and/or the second housing part.

2. Implantable medical device according to claim 1, wherein the slit has a constant height, wherein the height is defined as distance between the first housing part and the second housing part in a region in which the slit is formed.

3. Implantable medical device according to claim 1, wherein the slit has a varying height in a longitudinal direction of the slit, wherein the height is defined as distance between the first housing part and the second housing part in a region in which the slit is formed.

4. Implantable medical device according to claim 3, wherein the height of the slit decreases from a frontal opening of the slit to a bottleneck section of the slit.

5. Implantable medical device according to claim 4, wherein the bottleneck section is positioned, in a longitudinal direction of the slit, distally of the frontal opening of the slit and proximally of a distal terminus of the slit.

6. Implantable medical device according to claim 4, characterize a wall of the first housing part facing the slit and a wall of the second housing part facing the slit contact each other in the bottleneck section.

7. Implantable medical device according to claim 4, wherein a first barbed hook is formed in the bottleneck section.

8. Implantable medical device according to claim 1, wherein the first housing part and/or the second housing part comprises a recess for receiving a thread, wherein the recess is formed as an undercut in the longitudinal direction adjacent to the constriction so that a thread that is guided around the constriction can be slid into the recess.

9. Implantable medical device according to claim 8, wherein the first housing part and/or the second housing part comprises a second barbed hook that is formed between the constriction and the recess.

10. Implantable medical device according to any claim 1, wherein the first housing part and the second housing part are formed from one piece.

11. Implantable medical device according to claim 1, wherein the second housing part is mounted onto the first housing part.

12. Implantable medical device according to claim 1, wherein at least one of the first housing part and the second housing part are made from a metal, a plastic and/or a ceramic.

13. Implantable medical device according to claim 11, characterized wherein the second housing part is made from at least one material of the group consisting of liquid crystalline polymers, polysulfone, polyetherimides, polyether ether ketone, polyphenylsulfone, epoxy resin, titanium, and stainless steel.

14. Arrangement of an implantable medical device according to claim 1 and a thread for fixing the implantable medical device to a patient's body structure.

15. Method for fixing an implantable medical device according to claim 1 to a patient's body structure, the method comprising the following steps:
a) guiding a thread through a slit formed in a housing of the implantable medical device, wherein the slit separates the housing into a first housing part and a second housing part;
b) guiding the thread around a constriction formed in at least one of the first housing part and the second housing part;
c) forming a sling with the thread so that the sling extends around the first housing part and/or the second housing part in a region of the constriction; and
d) securing the thread to a body structure of a patient.
